(19) United States
(12) Patent Application Publication
(10) Pub. No.: US 2007/0119462 A1
(43) Pub. Date: May 31, 2007

(54) Arm protector

(76) Inventors: Donald Shumate SR., Oceanside, CA (US); Wendy Shumate, Oceanside, CA (US)

Correspondence Address: JAMES RAY & ASSOCIATES, 2640 PITCAIRN ROAD, MONROEVILLE, PA, 15146, US

(21) Appl. No.: 11/605,972

(22) Filed: Nov. 29, 2006

Related U.S. Application Data
(60) Provisional application No. 60/740,433, filed on Nov. 29, 2005.

(51) Int. Cl. A61F 5/37

(52) U.S. Cl. 128/846

(57) ABSTRACT

An apparatus for protecting against subcutaneous bleeding and skin tears in persons taking medications for thinning their blood includes an inner sleeve portion extending from a wrist of a wearer for a length of at least a forearm of such wearer. An outer portion extending from behind such wearers knuckles to at least such forearm for providing protection against such subcutaneous bleeding and skin tears in persons taking medications for thinning their blood. A strap is attached to such outer leather portion and extends between a thumb and index finger of such wearer. A securing device engages the strap and an outer surface of such outer leather portion for securing the apparatus in place and for enabling a portion of the apparatus to be turned back so that such wearer can wash their hands without removing the apparatus.

### CROSS REFERENCE TO RELATED APPLICATION

This patent application is related to and claims priority from U.S. Provisional Patent Application Ser. No. 60/______ filed Nov. 29, 2005.

### FIELD OF THE INVENTION

The present invention relates, in general, to arm protection type devices and, more particularly, this invention relates to an apparatus for protecting the arms of individuals who are required to take blood thinners for health reasons against undesirable bruises caused by thin blood.

### BACKGROUND OF THE INVENTION

Prior to the conception and development of the present invention, as is generally well known in the prior art, people on blood thinners are quite susceptible to subcutaneous bleeding and skin tears on their extremities when slight contact is made with their skin due to their age and certain types of medical complications. Such subcutaneous bleeding and skin tears are normally quite unsightly and are difficult to prevent. Nevertheless those persons who have heart trouble or are a candidate for a stroke must remain on such blood thinners.

Applicants are aware of several prior art type arm protectors which have been taught in the prior art. See, for example, U.S. Pat. No. 4,967,419 which teaches an arm protector for mechanics or other workers. U.S. Pat. No. 5,070,541 which teaches a protective covering for the back of the hand and forearm for protecting them while gathering eggs to prevent hens from pecking them. U.S. Pat. No. 6,854,130 teaches a protective garment an extremity covering piece attachable to a torso covering piece. None of these prior art protectors are designed to be attractive and have not seen any success in the market place with respect to persons who need protection against bumping portions of their bodies against something which causes bruising.

Applicants are also aware of U.S. Pat. Nos. 5,878,435 and 6,430,744 which teaches forearm type chaps as hand and forearm protectors. Each of these patents, however is not form fitting or attractive. Additionally, they require the covering to extent past the set of joints called the metacarpals and the phalanges because they are primarily designed to be worn by athletes for protection. Normally, such athletes are not prone to having heart attacks or strokes and they would certainly not be taking blood thinners.

### SUMMARY OF THE INVENTION

The present invention provides, according to a first embodiment, an apparatus for protecting against subcutaneous bleeding and skin tears in persons required to take certain types of medications for thinning their blood. Such apparatus includes an inner sleeve portion. The inner sleeve portion is formed from a first predetermined material. Such inner sleeve portion extends from a wrist of a wearer for a predetermined length of at least to an end of a forearm of such wearer. There is an outer portion formed from a second predetermined material, extending from a predetermined distance from behind such wearer's knuckles to such at least the end of such forearm of such wearer for providing such protection against subcutaneous bleeding and skin tears in such persons required to take such certain types of medications for thinning their blood. A strap like member, formed from a third predetermined material, is attached to such outer portion and extends between a thumb and index finger of such wearer. The final essential component of the apparatus is a means engageable with each of such strap and an outer surface of such outer portion for securing the apparatus in place and for enabling a portion of such apparatus to be turned back so that such wearer can wash their hands without having to remove the apparatus.

In a second embodiment the present invention provides an apparatus for protecting against subcutaneous bleeding and skin tears in persons required to take certain medications for thinning their blood. In this presently preferred embodiment such apparatus includes a first member fabricated from a first predetermined material having a thickness at least sufficient to protect predetermined areas of an arm and hand of such person's skin from bruising. The first member has a first predetermined shape and a first predetermined length which is at least sufficient to cover a back of such person's hand and extend over their forearm. Such apparatus also includes a second member having a predetermined amount of elasticity connected along two respective edges thereof to two respective edges of such first member for conforming the apparatus to such predetermined areas of such arm and hand of such person's skin to be protected.

### OBJECTS OF THE INVENTION

It is, therefore, one of the primary objects of the present invention to provide an apparatus for protecting certain extremities against undesirable subcutaneous bleeding and skin tears of an individual required to be on blood thinners for medical reasons.

Another object of the present invention is to provide an apparatus for protecting certain extremities against subcutaneous bleeding and skin tears of an individual required to be on blood thinners for medical reasons which is attractive.

Still another object of the present invention is to provide an apparatus for protecting certain extremities against subcutaneous bleeding and skin tears of an individual required to be on blood thinners for medical reasons which does not have to be removed to allow such individual to wash their hands.

Yet another object of the present invention is to provide an apparatus for protecting certain extremities against subcutaneous bleeding and skin tears of an individual required to be on blood thinners for medical reasons which is relatively easy to use.

An additional object of the present invention is to provide an apparatus for protecting certain extremities against subcutaneous bleeding and skin tears of an individual required to be on blood thinners for medical reasons which is relatively cool to wear.

Yet still another object of the present invention is to provide an apparatus from protecting certain extremities against subcutaneous bleeding and skin tears of individuals required to be on blood thinners for medical reasons which can be worn over previously bruised skin.

In addition to the various objects and advantages of the present invention described with some degree of specificity above it should be obvious that additional objects and advantages of the present invention will become more readily apparent to those persons who are skilled in the relevant art from the following more detailed description of the invention, particularly, when such detailed description is taken in conjunction with the attached drawing figures and with the appended claims.

### BRIEF DESCRIPTION OF THE DRAWINGS

FIG. 1 is a perspective view of the apparatus according to a first embodiment of the present invention illustrating an outer portion of the apparatus.

FIG. 2 is a perspective view of the apparatus shown in FIG. 1 which illustrates the inner portion of such apparatus; and

FIG. 3 is view illustrating a possible decorative nature of the present invention according to a presently preferred embodiment.

### BRIEF DESCRIPTION OF A PRESENTLY PREFERRED AND VARIOUS ALTERNATIVE EMBODIMENTS OF THE INVENTION

Prior to proceeding to the more detailed description of the present invention it should be noted that, for the sake of clarity and understanding, identical components which have identical functions have been identified with identical reference numerals throughout the several views illustrated in the drawing figures.

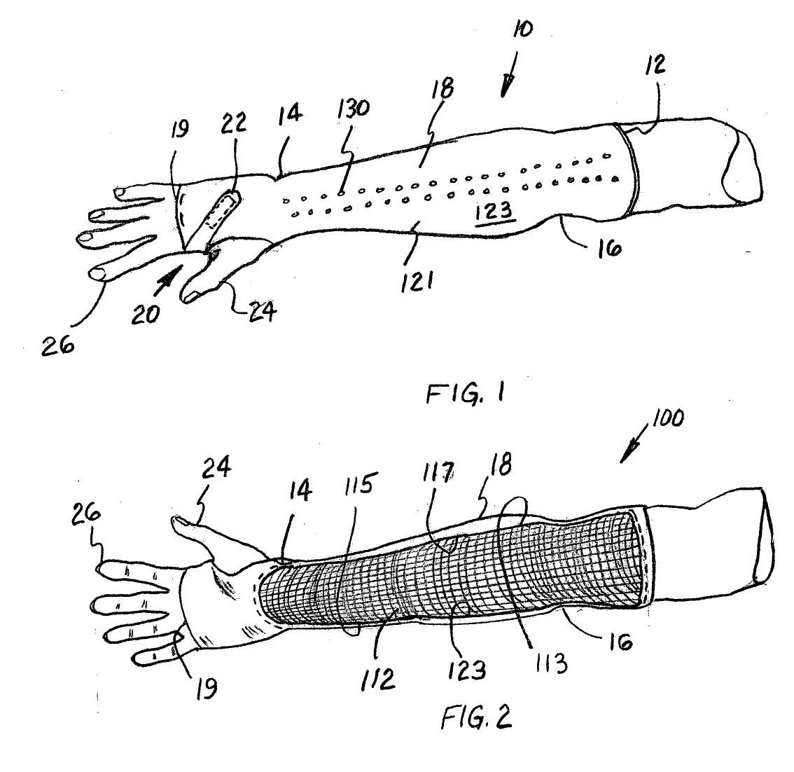

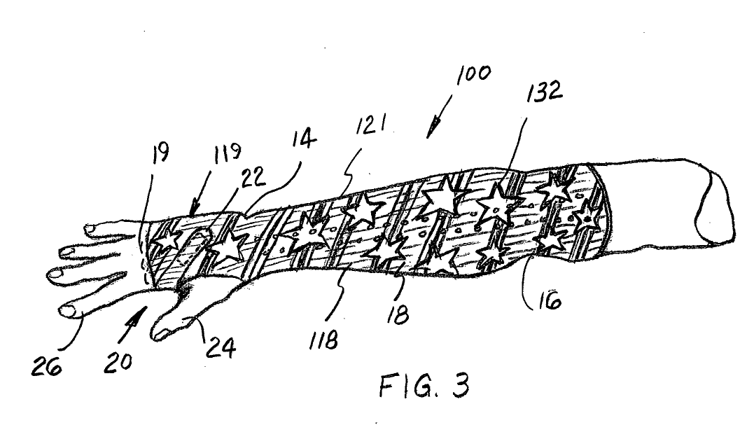

Reference is now directed, more particularly, to drawing FIGS. 1-3. Illustrated therein is an apparatus, generally designated 10, for protecting against subcutaneous bleeding and skin tears (not shown) in persons required to take certain types of medications for thinning their blood.

Such apparatus 10 includes, in a first embodiment thereof, an inner sleeve portion 12. The inner sleeve portion 12 is formed from a first predetermined material which extends from a wrist 14 of a wearer for a predetermined length to at least an elbow 16 of such wearer. In the presently preferred embodiment of the invention the predetermined material that such inner sleeve portion 12 is manufactured from is an elastic type material.

The apparatus 10 further includes an outer portion 18 which is formed from a second predetermined material. The outer portion 18 extends from a first predetermined distance located behind such wearer's knuckles 19 to at least such elbow 16 of such wearer in order to provide such protection against the unwanted subcutaneous bleeding and skin tears in those persons who are required to take such certain types of medications for medical reasons to thin their blood. Such second predetermined material that the outer portion 18 is manufactured from will be selected from the group consisting of leather and vinyl. In the presently preferred embodiment such outer portion 18 is formed from leather.

Apparatus 10 further includes a strap like member 22 which is formed from a third predetermined material. Such strap like member 22 is attached to such outer portion 18 and is disposed between a thumb 24 and index finger 26 of such wearer. The third predetermined material such strap like member 22 is manufactured from is also selected from the group consisting of leather and vinyl. Preferably such third predetermined material is leather.

In this embodiment of the invention there is a means, generally designated 20 engageable with each of such strap like member 22 and an outermost surface 28 of such outer leather portion 18 for securing the apparatus 10 in place and for enabling a portion of such apparatus 10 to be turned back so that such wearer can wash their hands without removing the apparatus 10.

According to the invention such means 20 for securing the apparatus 10 in place is one of a hook and loop type fastener (velcro), snaps, buttons and a buckle. It is presently preferred that such means 20 will be the hook and loop type fastener.

In the presently most preferred embodiment of the invention such inner sleeve portion 12 extends past such wearer's elbow 16 for a second predetermined distance. Further, in this embodiment such outer leather portion 18 extends past such wearer's elbow 16 for a third predetermined distance. Preferably, such second predetermined distance and such third predetermined distances are substantially identical.

According to a second embodiment of the present invention there is provided an apparatus, generally designated 100, for protecting against subcutaneous bleeding and skin tears in persons who for medical reasons are required to take certain medications for thinning their blood. Such apparatus 10 includes an first member 118 fabricated from a first predetermined material having a thickness at least sufficient to protect predetermined areas of an arm and hand of such person's skin from bruising, said first member 118 having a first predetermined shape and a first predetermined length which is at least sufficient to cover a back of such persons hand 119 and extend over their forearm 121.

There is a second member 112 having a predetermined amount of elasticity connected along two respective edges 113 and 115 thereof to two respective edges 117 and 123 of first member 118 for conforming the apparatus 100 to such predetermined areas of such arm 121 and hand 119 of such person's skin to be protected.

It is presently preferred that such first member 118 includes a plurality of apertures 130 formed therethrough to allow air to contact such skin of such wearer.

Additionally, such apparatus 100 further includes a strap like member 22 attached to the first member 118. There is a means 20 engageable with each of such strap like member 22 and an outer surface 123 of said first member 118 for securing said apparatus 100 to such wearer. Preferably, such means 20 for securing the apparatus 100 to such wearer is a hook and loop type fastener.

In the presently preferred embodiment of the invention such apparatus 100 further includes a decorative styling 132 formed over an outer surface 123 thereof which decorative styling 132 can be provided by one of various colors, fabrics and various combinations thereof. The second member 112 is attached to such first member 118 by sewing. Apparatus 100 can be safely worn on previously bruised skin.

While a presently preferred and various alternative embodiments of the present invention have been described in sufficient detail above to enable a person skilled in the relevant art to make and use the same it should be obvious that various other adaptations and modifications can be envisioned by those persons skilled in such art without departing from either the spirit of the invention or the scope of the appended claims.

We claim:

1. An apparatus for protecting against subcutaneous bleeding and skin tears in persons required to take certain types of medications for thinning their blood, said apparatus comprising:
(a) an inner sleeve portion, formed from a first predetermined material, extending from a wrist of a wearer for a predetermined length to at least an elbow of such wearer;
(b) an outer portion, formed from a second predetermined material, extending from a first predetermined distance from behind such wearer's knuckles to said at least such forearm of such wearer for providing said protection against said subcutaneous bleeding and skin tears in such persons required to take such certain types of medications for thinning their blood;
(c) a strap like member, formed from a third predetermined material, attached to said outer portion and extendable between a thumb and index finger of such wearer; and
(d) a means engageable with each of said strap and an outer surface of said outer leather portion for securing said apparatus in place and for enabling a portion of said apparatus to be turned back so that such wearer can wash their hands without removing said apparatus.

2. An apparatus, according to claim 1, wherein said first predetermined material said inner sleeve is made from is elastic.

3. An apparatus, according to claim 1, wherein said second predetermined material said outer portion is made from is selected from the group consisting of leather and vinyl.

4. An apparatus, according to claim 3, wherein said third predetermined material said strap like member is made from is selected from the group consisting of leather and vinyl.

5. An apparatus, according to claim 4, wherein said second and said third predetermined material is leather.

6. An apparatus, according to claim 1, wherein said means for securing said apparatus in place is one of a hook and loop type fastener, snaps, buttons and a buckle.

7. An apparatus, according to claim 6, wherein said means for securing said apparatus in place is a hook and loop type fastener.

8. An apparatus, according to claim 1, wherein said inner sleeve portion extends past such wearer's elbow for a second predetermined distance.

9. An apparatus, according to claim 8, wherein said outer portion extends past such wearer's elbow for a third predetermined distance.

10. An apparatus, according to claim 9, wherein said second predetermined distance and said third predetermined distance are substantially identical.

11. An apparatus for protecting against subcutaneous bleeding and skin tears in persons required to take certain medications for thinning their blood, said apparatus comprising:
(a) a first member fabricated from a first predetermined material having a thickness at least sufficient to protect predetermined areas of an arm and hand of such person's skin from bruising, said first member having a first predetermined shape and a first predetermined length which is at least sufficient to cover a back of such persons hand and extend over their forearm; and
(b) a second member having a predetermined amount of elasticity connected along two respective edges thereof to two respective edges of said first member for conforming said apparatus to such predetermined areas of such arm and hand of such person's skin to be protected.

12. An apparatus, according to claim 11, wherein said apparatus further includes plurality of apertures formed therethrough to allow air to contact such skin of such wearer.

13. An apparatus, according to claim 12, wherein said plurality of apertures are formed through said first member.

14. An apparatus, according to claim 11, wherein said apparatus further includes a strap like member attached to said first member.

15. An apparatus, according to claim 14, wherein said apparatus further includes a means engageable with each of said strap like member and an outer surface of said first member for securing said apparatus to such wearer.

16. An apparatus, according to claim 15, wherein said means for securing said apparatus to such wearer is a hook and loop type fastener.

17. An apparatus, according to claim 11, wherein said apparatus further includes a decorative styling formed over an outer surface thereof.

18. An apparatus, according to claim 17, wherein said decorative styling can be provided by one of various colors, fabrics and various combinations thereof.

19. An apparatus, according to claim 11, wherein said apparatus can be safely worn on previously bruised skin.

20. An apparatus, according to claim 11, wherein said second member is attached to said first member by sewing.
